(19) United States
(12) Patent Application Publication
(10) Pub. No.: US 2024/0307312 A1
(43) Pub. Date: Sep. 19, 2024

(54) FORMULATIONS OF ACTIVE PHARMACEUTICAL INGREDIENTS (APIs) FOR LOCAL DELIVERY VIA ACTIVE MICROBOT

(71) Applicants: Alex KISELYOV, San Diego, CA (US); BIONAUT LABS LTD., Herzliya (IL)

(72) Inventor: Alex KISELYOV, San Diego, CA (US)

(73) Assignee: BIONAUT LABS LTD., Herzliya (IL)

(21) Appl. No.: 18/571,184

(22) PCT Filed: Jun. 13, 2022
(86) PCT No.: PCT/US22/33256

§ 371 Date: Dec. 15, 2023

Related U.S. Application Data
(60) Provisional application No. 63/213,945, filed on Jun. 23, 2021.

(51) Int. Cl. A61K 9/20; A61J 3/10; A61K 9/28; A61K 45/06

(52) U.S. Cl. CPC A61K 9/2072; A61J 3/10; A61K 9/205; A61K 9/2095; A61K 9/28; A61K 45/06

(57) ABSTRACT

Provided herein are pharmaceutical formulations, methods of manufacturing thereof, and methods of treatment. The pharmaceutical formulations are adapted to serve as a pay load on or in a microbot, and comprise a tablet of a therapeutically effective dose of a pharmaceutical agent and have dimensions such that the tablet may fit on or in the microbot. The pharmaceutical formulation may be formed into, e.g., a cylinder or spheroid of 1 mm dimension, and the pharmaceutical agent may be a small molecule drug, a peptide, a peptoid, a biologic drug, cells, or any combination thereof. Thus, the pharmaceutical formulation of the present description offers delivery of targeted therapy at a locus in a subject, e.g., a tumor.

### FIELD OF INVENTION

The present invention relates generally to the field of pharmaceutical formulations, delivery systems, and methods.

### BACKGROUND

Therapeutic agents are traditionally administered to subjects using various routes and delivery means. Therapeutic delivery means include, for example, ingestible pill or capsule, powder, syrup, salve or cream, gel, hypodermic syringe, nebulized spray, aqueous solution, non-aqueous solution, suppository, or transdermal patch. Each of these traditional delivery means provides certain advantages and disadvantages.

None of the traditional delivery means, however, is well suited for localized delivery of a therapeutic agent inside a subject's body. Often, due to risk of adverse side effects from global administration throughout a subject's body, it would be preferable to deliver a therapeutic only to a desired target such as, for example, a tumor. Further, some therapeutic agents are very expensive, and it would be more efficient use of a valuable resource to target the expensive therapeutic only to where it is needed in a patient's body. In many medical applications, it would be useful to use a mobile medical device to move within a living organism. For example, it may be desirable to move an internal device through tissue to a particular desired anatomic location to release a therapeutic in a controlled manner.

Additionally, bioavailability and pharmacokinetics may make it very challenging to sustain an ideal effective dosage in a patient, especially proximate to a localized delivery target such as a tumor. The patient's body may metabolize or eliminate therapeutic agents, causing the levels of such therapeutic agents to fluctuate and decline. It would be desirable to have systems and methods that would permit on-demand release of therapeutic agents at preferred times in preferred locations in a patient.

Further, traditional therapeutics routes and delivery means require a patient or medical professional to actively follow a dosing regimen. This could include, e.g., taking pills correctly in the right quantity and timing, or correctly dosing and administering an injectable biologic. Some therapeutics may require repeated doctor's visits. There may be various incidental dosing instructions, especially for ingestible drugs, such as avoiding certain foods, such as citrus. These dosing regimens can pose significant problems for patients with limited ability to care for themselves, including, e.g., children and dementia patients. Compliance with dosing regimens can be a burdensome problem for any adult, who may simply forget or may be unable to travel easily to make a doctor's appointment. It would be highly desirable to have systems and methods that could reduce or eliminate the need for people to manage regimen instructions.

Also, many traditional therapeutic administration routes and means are painful or uncomfortable for the patient. Reducing, e.g., the frequency and duration of hypodermic injections or intravenous infusions would offer substantial improvement to a patient's comfort and quality of life.

This disclosure provides pharmaceutical formulations designed to alleviate these shortcomings of traditional delivery of therapeutic agents. Pharmaceutical formulations described herein offer local drug release, which can limit adverse effects and preserve scarce and/or expensive therapeutic agents. The therapeutic agents may be delivered and released locally, at preferred times, and in a gradual or long-term release regimen, which enhances clinicians' control over the dose and rate of exposure, and may reduce the need for frequent exposure, such as by repeated injections or long-term intravenous drip, by providing a longer duration of sustained therapy.

### BRIEF SUMMARY

The present disclosure provides pharmaceutical formulations adapted to be payload on or in a microbot, methods of manufacturing such pharmaceutical formulations, and methods of treatment. In an embodiment, the present disclosure provides a pharmaceutical formulation adapted to be carried as payload in or on a microbot, comprising: a tablet of a therapeutically effective dose of a pharmaceutical agent and having dimensions such that the tablet may fit on or in the microbot.

In any embodiment, the pharmaceutical agent may be a small molecule drug, a peptide, a peptoid, or a biologic drug, or any combination thereof. In any embodiment, the pharmaceutical agent may be a biologic drug. In any embodiment wherein the pharmaceutical agent is a biologic drug, the biologic drug may be suspended in a hydrogel matrix. In any aspect, the hydrogel matrix may consist of a cross-conjugated homocysteine-norbornene complex, or alternatives based on ‘click’-chemistry. In any aspect the biologic drug may be suspended in an isotonic lyophilized polyplex. In any aspect, the isotonic lyophilized polyplex may be selected from the group consisting of: poly-L-lysine, phosphorylcholine-modified polyethyleneimine, and PEGylated polyethyleneimine.

In any embodiment, the pharmaceutical agent may comprise one or more radiosensitizers. In any embodiment, the pharmaceutical agent may consist of any one or more of doxorubicin, topotecan, and temozolomide.

In any embodiment of the pharmaceutical formulation, the pharmaceutical agent may comprise about 1 to about 3,000 μg aggregate mass per tablet.

In any embodiment, the tablet may be formulated by wet granulation, dry granulation, or melt extrusion.

In any embodiment, the tablet may be adapted to fit in or on the microbot. In any aspect or embodiment, the tablet may be substantially cylindrical or substantially spheroid. In any aspect or embodiment, the tablet may be substantially cylindrical. In any aspect or embodiment, the tablet may be substantially spheroid. In any aspect or embodiment, the tablet may have a length/major axis dimension of about 2 mm and a diameter/minor axis dimension of about 1 mm. In any aspect or embodiment, the tablet may have a length/major axis dimension of about 1 mm and an average diameter/minor axis dimension of about 0.85 mm.

In any aspect or embodiment, the tablet may further comprise at least one pharmaceutically acceptable binder, carrier, and/or excipient. In any embodiment, the at least one pharmaceutically acceptable binder, carrier, and/or excipient may be selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone. In any embodiment, the tablet may further comprise a pharmaceutically acceptable coating.

In any embodiment, the pharmaceutical formulation may conform to USP905 consistent uniformity requirements.

In an aspect, the present disclosure provides a therapeutic delivery system comprising a microbot having a dimension ranging from 50 nm to 1 cm, and having a motility component, and carrying a therapeutic cargo comprising a pharmaceutical formulation adapted to be carried as payload in or on the microbot, the pharmaceutical formulation comprising: a tablet a therapeutically effective dose of a pharmaceutical agent and having dimensions such that the tablet may fit on or in the microbot.

In an aspect, the present disclosure provides a method of manufacturing a tablet adapted to serve as a cargo on a microbot, comprising the steps of: forming a premix, the premix comprising a mixture comprising a therapeutically effective dose of a pharmaceutical agent; and shaping the premix into a tablet having dimensions such that the tablet may fit on or in the microbot.

In any embodiment of the method of manufacturing, the shaping may be achieved by wet granulation, dry granulation, or melt-extrusion. In any embodiment of the method of manufacturing, the pharmaceutical agent may be a small molecule drug, a peptide, a peptoid, or a biologic drug, or any combination thereof. In any embodiment of the method of manufacturing, the pharmaceutical agent may be a biologic drug. In any embodiment of the method of manufacturing, the biologic drug may be suspended in a hydrogel matrix. In any embodiment of the method of manufacturing, the biologic drug may be suspended in an isotonic lyophilized polyplex. In any embodiment of the method of manufacturing, the isotonic lyophilized polyplex may be selected from the group consisting of: poly-L-lysine, phosphorylcholine-modified polyethyleneimine, and PEGylated polyethyleneimine.

In any embodiment of the method of manufacturing, the hydrogel matrix may be based on ‘click’ chemistry. In any embodiment of the method of manufacturing, the pharmaceutical agent may be a radiosensitizer. In any embodiment of the method of manufacturing, the pharmaceutical agent may be selected from the list consisting of doxorubicin, topotecan, and temozolomide.

In any embodiment of the method of manufacturing, the pharmaceutical agent may comprise about 1 to about 3,000 μg aggregate mass per tablet. In any embodiment of the method of manufacturing, the tablet may be adapted to fit in the microbot. In any embodiment of the method of manufacturing, the tablet may be substantially cylindrical or substantially spheroid. In any embodiment of the method of manufacturing, the tablet may be substantially cylindrical. In any embodiment of the method of manufacturing, the tablet may be substantially spheroid. In any embodiment of the method of manufacturing, the tablet may have a length/major axis dimension of about 2 mm and a diameter/minor axis dimension of about 1 mm. In any embodiment of the method of manufacturing, the tablet may have a length/major axis dimension of about 1 mm and a diameter/minor axis dimension of about 0.85 mm.

In any embodiment of the method of manufacturing, the tablet may further comprise at least one pharmaceutically acceptable binder, carrier, and/or excipient. In any embodiment of the method of manufacturing, the at least one pharmaceutically acceptable binder, carrier, and/or excipient may be selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone. In any embodiment of the method of manufacturing, the tablet may further comprise a pharmaceutically acceptable coating material.

In any embodiment of the method of manufacturing, the tablet may conform to USP905 consistent uniformity requirements.

In an aspect, the present disclosure provides a method of manufacturing a tablet to be carried as payload in or on a microbot, comprising the steps of: (1) preparing a premix consisting of about 90 parts doxorubicine, about 450 parts hydrous lactose, about 60 parts polyvinyl alcohol, about 35 parts purified water, about 6 parts fumed silica, and about 6 parts magnesium stearate; (2) raising the premix to about 50° C. for a duration of about 16 hours; (3) passing the premix through a 30 mesh standard screen; and (4) pressing the premix into tablets.

In another aspect, the present disclosure provides another method of manufacturing a tablet to be carried as payload in or on a microbot, comprising the steps of: (1) preparing a premix consisting of about 444 parts doxodubicine, about 545 parts hydrous lactose, and about 10 parts magnesium stearate; (2) dry blending the premix; and (3) placing the premix into a tablet press configured to press spheroidal tablets having a length of about 1 mm and average diameter of about 0.85 mm.

In still another aspect, the present disclosure provides another method of manufacturing a tablet to be carried as payload in or on a microbot, comprising the steps of: (1) suspending the at least one biologic- or cell-based therapeutic in a hydrogel matrix, the hydrogel matrix comprising NorHA; and (2) packing the cargo into dimensions such that the tablet may fit on or in the microbot.

### BRIEF DESCRIPTION OF THE DRAWINGS

Exemplary embodiments of the invention will be better understood and readily apparent to one of ordinary skill in the art from the following written description, by way of example only, and in conjunction with the drawings, in which:

FIG. 1 depicts an exemplary schematic of a microbot, labeled in the schematic as a “Bionaut™ microrobot.”

FIG. 2 depicts exemplary schematics of pharmaceutical formulations that may be formed to be carried as payload on or in a microbot.

FIG. 3 depicts a table reflecting various pharmaceutical formulation manufacturing techniques, and their suitability for forming tablets comprising various therapeutic agents, and such therapeutic agents' estimated maximum allowable doses.

FIG. 4A is a scale photograph of various typical embodiments of tablets of varying dimensions. FIG. 4B is a photograph of cylindrical tablets having a diameter of about 0.85 mm and suitable for payload in a microbot.

FIG. 5 is a flow diagram depicting the steps of a method of manufacture of a tablet, as described in the present disclosure, which may be accomplished by any of wet granulation, dry granulation, and/or melt-extrusion.

FIG. 6 depicts schematics for the synthesis of norbornene-hyaluronic acid (NorHA) hydrogels. Part (a) depicts the synthesis of NorHA from hyaluronic acid-tetrabutylammonium (HA-TBA) via the coupling of norbornene carboxylic acid to pendant alcohols on HA. The reaction proceeds through a di-tert-butyl di-carbonate (Boc₂O) activated process, yielding NorHA, with 20 wt % of its repeat units functionalized with norbornene. Part (b), below, depicts a synthesis scheme for formation of gels through a light-initiated thiolene reaction, between a di-thiol crosslinker and NorHA, with subsequent chemical modification with mono-thiols and/or di-thiols.

FIG. 7A depicts a schematic flow diagram of a development and evaluation of a lyophilized plasmid/linear polyethyleneimine (LPEI) polyplex formulation, with long-term stability for microbot-mediated local delivery of biologic drugs such as oligonucleotides. FIG. 7B depicts a schematic flow diagram of a development and evaluation of a lyophilized, stable and active siRNA polyplex formulation for microbot-mediated local delivery of biologic drugs such as oligonucleotides.

### DETAILED DESCRIPTION OF THE INVENTION

As used herein, including the appended claims, the singular forms of words such as “a,” “an,” and “the,” shall include their corresponding plural references, unless context clearly dictates otherwise.

All references cited herein are incorporated by reference to the same extent as if each individual publication, patent application, or patent, was specifically and individually indicated to be incorporated by reference.

For many medical interventions, it would be desirable to have compositions, systems, and methods for deploying a therapeutic agent in a directed way within a living subject, such that the therapeutic agent may be delivered to a specific locus in the subject, such as, for example, a tumor.

The present disclosure provides a formulation of diverse therapeutic modalities, including small molecule, biologic drug, and cell-based therapies for the use in microbots, and which are designed for accurate, active delivery of therapeutic agents to specific loci of therapeutic interest. Such loci of interest include lesions, lumens, ventricles, tumors, ducts, vasculature, tissues, organs, layers, envelopes, physiological barriers, and any combination thereof.

The present disclosure provides a pharmaceutical formulation, delivery system, and method of manufacture thereof, wherein the pharmaceutical formulation comprises a tablet formulated for delivery on or in a microbot.

The microbot is a diagnostics- and/or therapeutics-bearing robot of less than 5 cm dimension (and as small as micron-scale dimension) designed to navigate with great precision of provide 3-dimensional control, following complex 3D trajectories in a subject's lumens, tissues, organs, etc. The microbot may be propelled by an external (i.e., remote) force, such as by permanent magnet(s), electromagnet(s), electrical, piezoelectric, ultrasound, optical, radiofrequency filed(s), and/or any combination thereof.

The microbot may be made from diverse materials exhibiting (a) wide range of stiffness properties (e.g., having Young's modulus of 0.1-100 GPa); (b) surface features (coatings, abrasives, adhesives, immobilized and/or absorbed materials, multiple layers, hydrophilic, hydrophobic, charged, neutral, enzymatically active); (c) shape (sphere, spheroid, cylinder, prism, screw, blade); (d) size (100 μM to 5 cm length, 50 μM to 5 mm outer diameter); and (e) compartments to accommodate (i) external propulsion force responding element, as exemplified by a magnet, and (ii) diagnostic and/or therapeutic payload. The diagnostics and therapeutic payloads may be specifically tailored for the microbot to diagnose and treat specific condition(s) in the loci of interest.

The modality (e.g., small molecule, biologics, cells), dosing, regimen, duration/frequency of treatment using microbot is determined from the respective microbot-mediated in vitro, ex vivo and in vivo studies that model the specific disease as well as from the respective clinical and preclinical literature. Specific experiments include but are not limited to the potency, selectivity, specificity, efficacy, pharmacokinetics, distribution, exposure, pharmacodynamics, and toxicity studies following the microbot-mediated local delivery of the active pharmaceutical or diagnostic ingredient.

A specific formulation may be selected using the aforementioned criteria, as well as the desired payload release mechanism, the dimensions of the therapeutic/diagnostic cargo compartment as well as sterilization process.

Representative formulation techniques for the microbot diagnostic and/or therapeutic cargo may include liquid, solid, and/or diverse colloidal systems, as exemplified by physiological solutions, mini-tablets, micro-tablets, micelles, liposomes, nanoparticles, beads, gels, and the like.

The microbot is a versatile “payload agnostic” suited for a wide variety of delivery modalities, each of which may be tailored to suit the therapeutic agent of interest. For instance, some therapeutics may be water-soluble and other therapeutics may be water insoluble. Some therapeutics may be stable and some may be sensitive to temperature, pH, and the like.

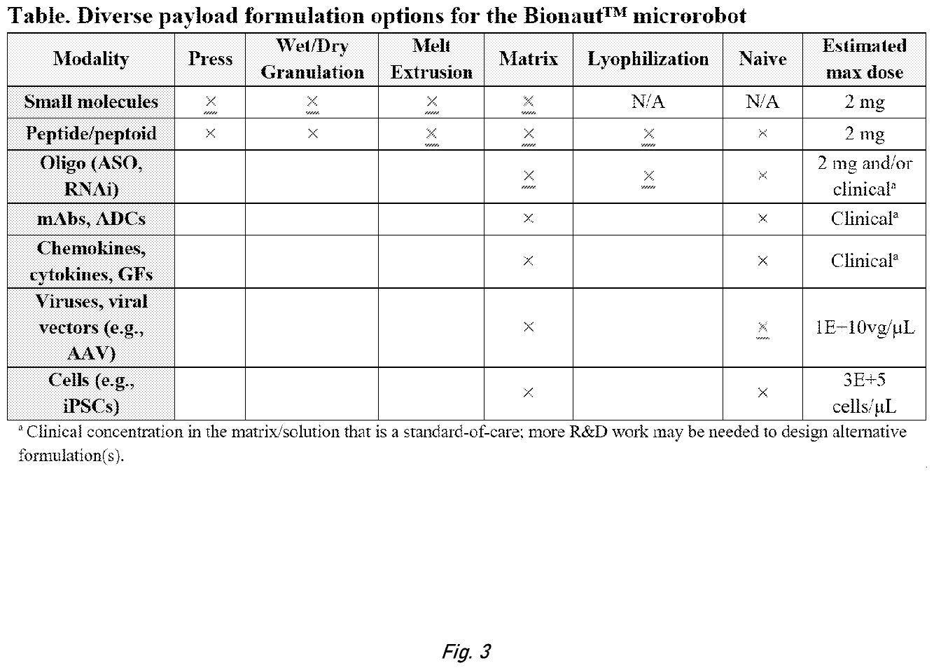

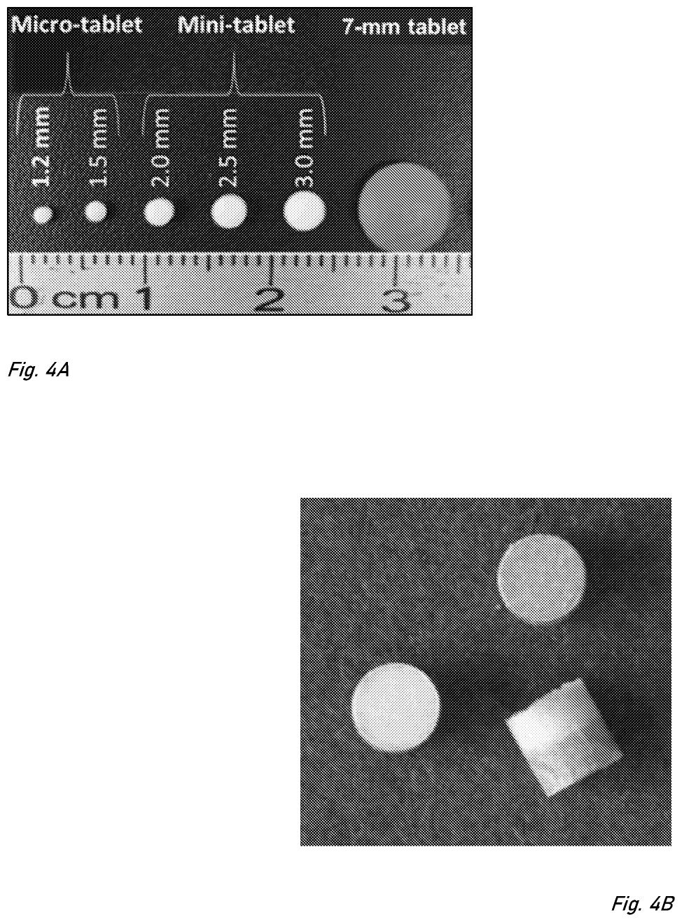

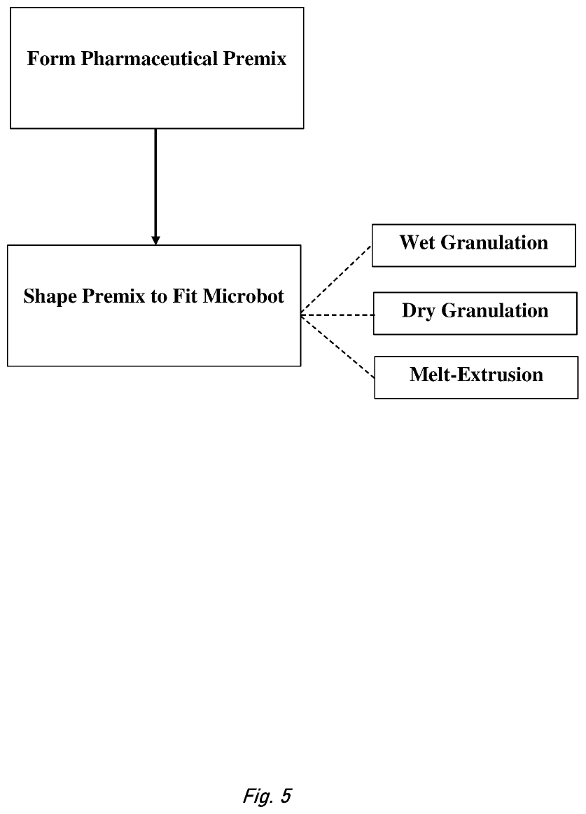

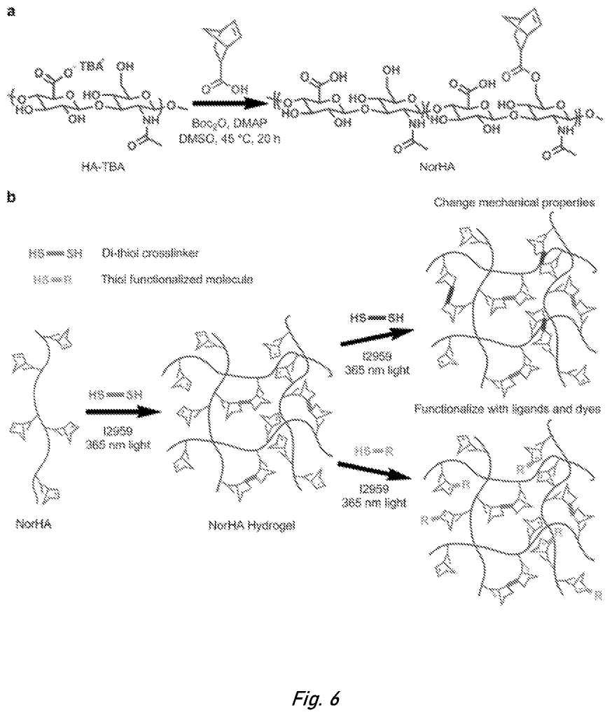

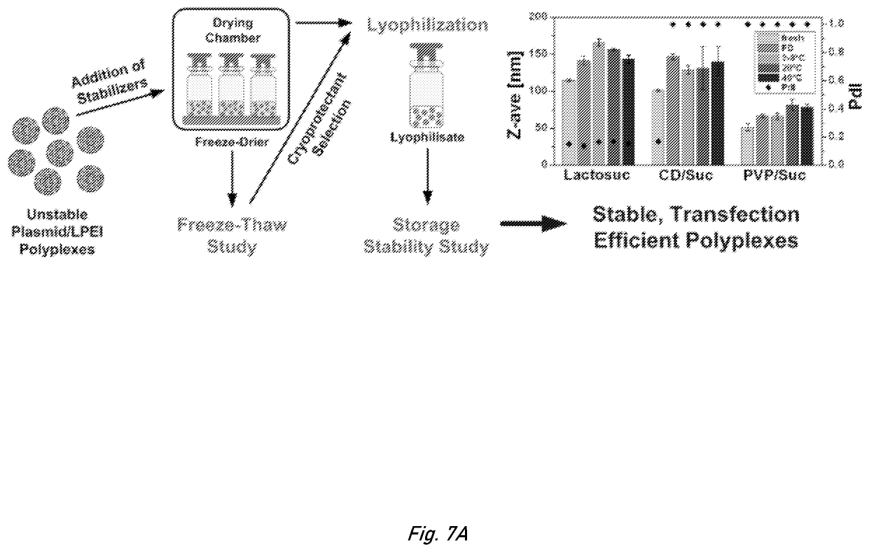

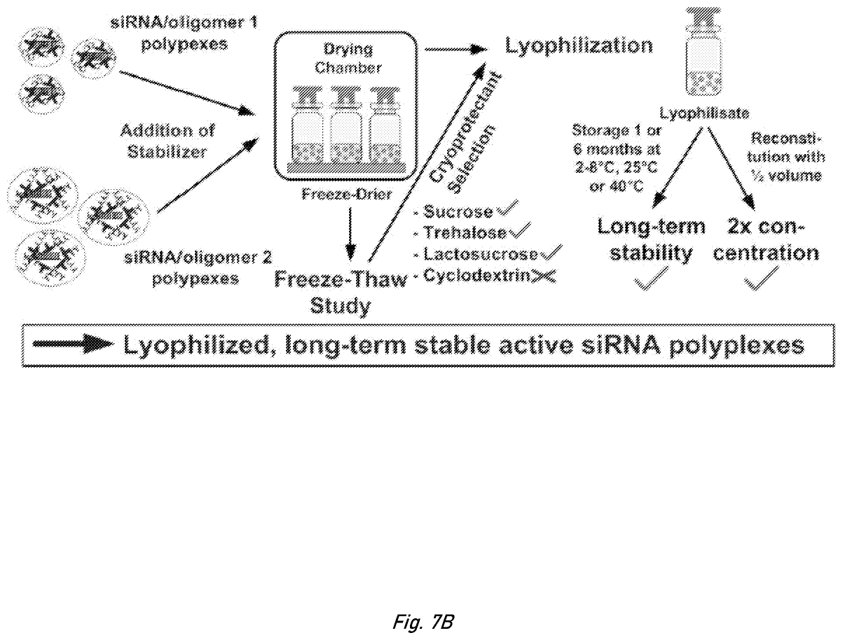

In general, small molecule-based and peptide/peptoid-based therapeutics offer great(er) flexibility with respect to available formulation options, because small molecule drugs tend to be reasonably stable and tolerate a variety of delivery mechanisms. Biologics-based products and cells, on the other hand, may require a particular delivery technique. Notably, for majority of biologics-based therapeutic modalities may require suspensions, buffers, temperature controls, etc. Some of these modalities and their payload formulation options are highlighted in a table presented in FIG. 3.

The present disclosure provides pharmaceutical formulations for delivery via microbot. One or more therapeutic agents may be delivered in the form of a tablet, e.g., a mini-tablet or a micro-tablet, formulated and shaped to fit as payload on or in the microbot. The microbot may be driven and directed (for example by way of electromagnetic force) through Newtonian or non-Newtonian media exemplified by serum, cerebrospinal fluid (CSF), elastic, viscoelastic or viscous biological media including tissue and organs, from an introduction site, e.g., an incision, to a target locus in a subject's body. The microbot could thereby deliver a therapeutic agent from an orifice or incision to a localized target such as a tumor. The microbot could be configured to accomplish immediate, delayed, controlled or slow release of the therapeutic agent. The tablet comprising the therapeutic agent may be fast-release (e.g., highly soluble or slow-release (e.g., slightly soluble), and the solubility characteristics may be tailored to suit the desired rate of release of therapeutic agent.

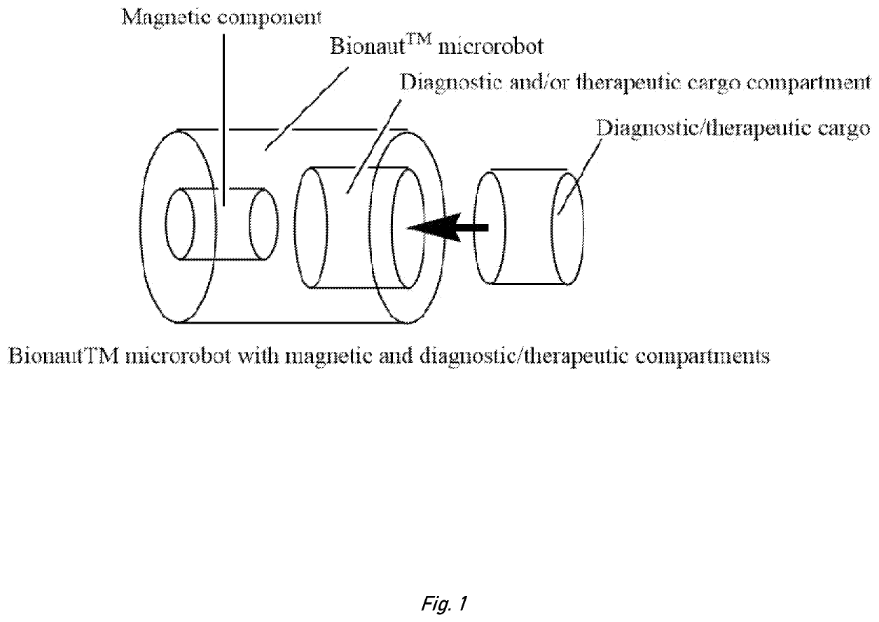

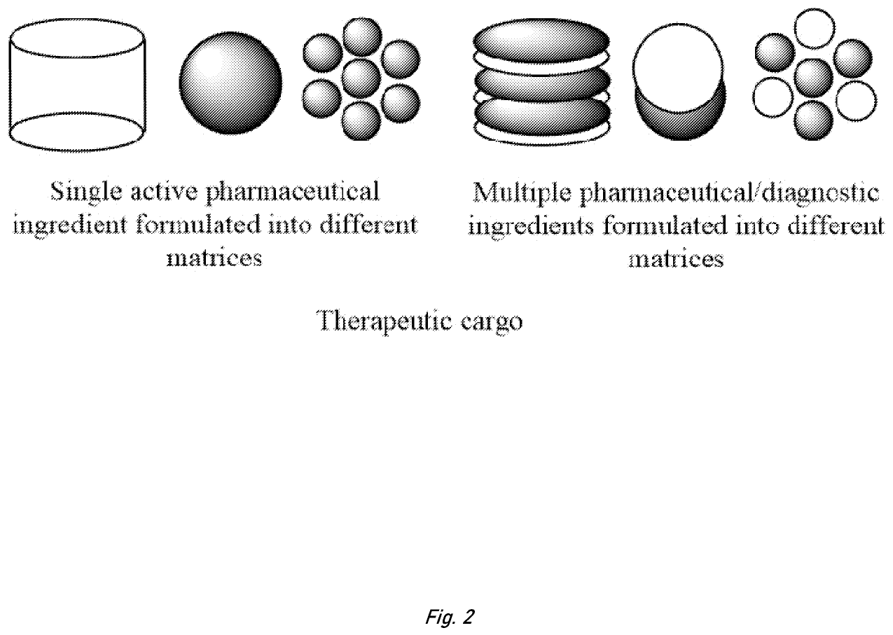

The microbot may comprise a miniature therapeutics delivery robot, FIG. 1, having a dimension of 50 nm to 1 cm, and which has a motility component. The motility component may be configured to drive and direct the delivery device by magnetic means. For example, the motility component may operate according to the disclosures of U.S. Ser. No. 16/609,493 and/or PCT/US2019/041309, which are incorporated by reference in their entireties. The delivery device may be controlled according to the disclosure of U.S. Ser. No. 16/620,748, which is incorporated by reference in its entirety. The microbot may delivery the pharmaceutical formulation(s) it carries in response to external stimuli, endogenous stimuli, or both. In any embodiment the delivery device may release the therapeutic cargo in response to endogenous stimuli (including, e.g., temperature, pH, RedOx signals, pressure, salinity, enzymes, biochemical gradients exemplified but not limited to chemokines and cytokines, receptors and/or agonists) within a subject. The subject may be any animal, including non-mammals, mammals, and human patients.

In general, the pharmaceutical formulation may be formulated into a tablet and the tablet placed on or in the microbot, as payload. The microbot may release the pharmaceutical formulation in response to an external force. The external force may be selected from mechanomagnetic, electromagnetic, piezoelectric, ultrasound, radiofrequency, optical treatment, or any combination thereof. For example, the pharmaceutical formulation may be released by exogenous ultrasound stimulus, according to the disclosures of PCT/US2018/030949 and/or U.S. Ser. No. 17/052,201, which are incorporated by reference in their entireties.

The microbot may comprise a motility component that drives the delivery device from an introduction site, e.g., an injection site, on a subject to a target locus within the subject. For instance, the microbot may be inserted via microcatheter into the patient's lumbar spine, and the motility component then drives the microbot to a locus in the patient's midbrain and presents the pharmaceutical formulation by releasing it. For another example, the microbot may be inserted into a vessel in a human patient's arm and the motility component drives the microbot to the patient's liver. For yet another example, the microbot may be inserted into the patient's esophagus and motility component drives the microbot to a lesion located on the inner lumen of the patient's duodenum. The motility component may propel the microbot by means of electromagnetism, ultrasound, radiofrequency, optical, electrical, alternative, or any combination thereof. In any embodiment, the microbot may be propelled from the injection site to the target locus through any of biological matrix, a tissue, an organ, circuitry, vessel(s), a lumen, or any combination thereof. The microbot may drive according to the disclosure of PCT/US2019/059178, which is incorporated by reference in its entirety. In any embodiment, the microbot may be repositioned or removed. More information regarding the deployment and retraction of the microbot may be found in PCT/US2019/030355, which is incorporated by reference in its entirety.

The pharmaceutical formulation may comprise a tablet, which may comprise any of a small molecule drug, a biologic drug, a microbe (i.e., bacteria, archaea, and/or single-celled eukaryote), virus, viral construct, micelle(s), nutrient, mineral, vitamin, peptide, peptoid, enzyme, antibody or antibody fragment (whether engineered or natural), nucleic acid (including DNA and/or RNA), carbohydrate, lipid or fatty acid, aqueous solution, non-aqueous solution, nanomaterial, nanoparticle (e.g., immunogold nanoparticle), glue, binder, chemical suture, label (e.g., radiolabel), or any combination thereof.

The present disclosure provides pharmaceutical formulations adapted to be payload on or in a microbot, methods of manufacturing such pharmaceutical formulations, and methods of treatment. In an embodiment, the present disclosure provides a pharmaceutical formulation adapted to be carried as payload in or on a microbot, comprising: a tablet a therapeutically effective dose of a pharmaceutical agent and having dimensions such that the tablet may fit on or in the microbot.

In any embodiment, the pharmaceutical agent may be a small molecule drug, a peptide, a peptoid, a biologic drug, or any combination thereof. In any embodiment, the pharmaceutical agent may be a biologic drug, e.g., a drug derived from a biological material including protein, antibody, antibody fragment, DNA, RNA, plasmid, etc. In any embodiment wherein the pharmaceutical agent is a biologic drug, the biologic drug may be suspended in a hydrogel matrix. In any aspect, the hydrogel matrix may consist of a cross-conjugated homocysteine-norbornene complex. In any aspect the biologic drug may be suspended in an isotonic lyophilized polyplex. In any aspect, the isotonic lyophilized polyplex may be selected from the group consisting of: poly-L-lysine, phosphorylcholine-modified polyethyleneimine, and PEGylated polyethyleneimine.

In any embodiment, the pharmaceutical agent may comprise one or more radiosensitizers. In any embodiment, the pharmaceutical agent may consist of any one or more of such radiosensitizers as doxorubicin, topotecan, and temozolomide.

In any embodiment of the pharmaceutical formulation, the pharmaceutical agent may comprise from about 1 μg up to about 1,000 μg aggregate mass per tablet. For example, the pharmaceutical agent may comprise 50 μg of radiosensitizer and 50 μg microtubule-targeting agent (MTA), providing a total of 100 μg aggregate mass of pharmaceutical agent. The pharmaceutical formulation may comprise about 1, 2, 3, 4, 5, 10, 15, 20, 50, 100, 200, 300, 400, 500, 600, 700, 800, 900, 1,000 and up to about 3,000 μg aggregate mass per tablet. In an embodiment, the pharmaceutical agent may comprise from about 3 μg up to 25 μg aggregate mass per tablet.

In any embodiment, the tablet may be formulated by wet granulation, dry granulation, or melt extrusion.

In any embodiment, the tablet may be adapted to fit in the microbot. In any embodiment of the method of manufacturing, the tablet may be cylindrical or substantially cylindrical. In any embodiment of the method of manufacturing, the tablet may be spheroid, substantially spheroid, or hemispheroid. In any aspect or embodiment, the tablet may have a length/major axis dimension of about 2 mm and a diameter/minor axis dimension of about 1 mm. In any aspect or embodiment, the tablet may have a length/major axis dimension of about 1 mm and an average diameter/minor axis dimension of about 0.85 mm. The length/major axis dimension may be 50 nm, 55 nm, 60 nm, 70 nm, 80 nm, 90 nm, 100 nm, 150 nm, 200 nm, 300 nm, 400 nm, 500 nm, 750 nm, 1000 nm (1 μm), 1.1 μm, 1.2 μm, 1.3 μm, 1.4 μm, 1.5 μm, 1.6 μm, 1.7 μm, 1.8 μm, 1.9 μm, 2.0 μm, 5.0 μm, 10 μm, 15 μm, 20 μm, 25 μm, 30 μm, 40 μm, 50 μm, 75 μm, 100 μm, 200 μm, 300 μm, 400 μm, 500 μm, 600 μm, 700 μm, 850 μm, 800 μm, 900 μm, 1000 μm (1 mm), 1.1 mm, 1.2 mm, 1.3 mm, 1.4 mm, 1.5 mm, 1.6 mm, 1.7 mm, 1.8 mm, 1.9 mm, 2.0 mm, 2.5 mm, 3.0 mm, 3.5 mm, 4.0 mm, 5.0 mm, 6.0 mm, 7.0 mm, 8.0 mm, 9.0 mm, or 10.0 mm (1 cm). The width/diameter/minor axis dimension may be 50 nm, 55 nm, 60 nm, 70 nm, 80 nm, 90 nm, 100 nm, 150 nm, 200 nm, 300 nm, 400 nm, 500 nm, 750 nm, 1000 nm (1 μm), 1.1 μm, 1.2 μm, 1.3 μm, 1.4 μm, 1.5 μm, 1.6 μm, 1.7 μm, 1.8 μm, 1.9 μm, 2.0 μm, 5.0 μm, 10 μm, 15 μm, 20 μm, 25 μm, 30 μm, 40 μm, 50 μm, 75 μm, 100 μm, 200 μm, 300 μm, 400 μm, 500 μm, 600 μm, 700 μm, 800 μm, 850 μm, 900 μm, 1000 μm (1 mm), 1.1 mm, 1.2 mm, 1.3 mm, 1.4 mm, 1.5 mm, 1.6 mm, 1.7 mm, 1.8 mm, 1.9 mm, 2.0 mm, 2.5 mm, 3.0 mm, 3.5 mm, 4.0 mm, 5.0 mm, 6.0 mm, 7.0 mm, 8.0 mm, 9.0 mm, or 10.0 mm (1 cm).

In any aspect or embodiment, the tablet may further comprise at least one pharmaceutically acceptable binder, carrier, and/or excipient. In any embodiment, the at least one pharmaceutically acceptable binder, carrier, and/or excipient may be selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone. In any embodiment, the tablet may further comprise a pharmaceutically acceptable coating.

In any embodiment, the pharmaceutical formulation may conform to USP905 consistent uniformity requirements.

In an aspect, the present disclosure provides therapeutic delivery systems comprising a microbot having a dimension ranging from 50 nm to 1 cm, and having a motility component, and carrying a therapeutic cargo comprising a pharmaceutical formulation adapted to be carried as payload in or on the microbot, the pharmaceutical formulation comprising: a tablet a therapeutically effective dose of a pharmaceutical agent and having dimensions such that the tablet may fit on or in the microbot.

In an aspect, the present disclosure provides methods of manufacturing a tablet adapted to serve as a cargo on a microbot, comprising the steps of: forming a premix, the premix comprising a mixture comprising a therapeutically effective dose of a pharmaceutical agent; and shaping the premix into a tablet having dimensions such that the tablet may fit on or in the microbot.

In any embodiment of the method of manufacturing, the shaping may be achieved by wet granulation, dry granulation, or melt-extrusion. In any embodiment of the method of manufacturing, the pharmaceutical agent may be a small molecule drug, a peptide, a peptoid, or a biologic drug, or any combination thereof. In any embodiment of the method of manufacturing, the pharmaceutical agent may be a biologic drug. In any embodiment of the method of manufacturing, the biologic drug may be suspended in a hydrogel matrix. In any embodiment of the method of manufacturing, the biologic drug may be suspended in an isotonic lyophilized polyplex. In any embodiment of the method of manufacturing, the isotonic lyophilized polyplex may be selected from the group consisting of: poly-L-lysine, phosphorylcholine-modified polyethyleneimine, and PEGylated polyethyleneimine.

In any embodiment of the method of manufacturing, the hydrogel matrix may be based on ‘click’ chemistry, as exemplified, but not limited to alkyne-azide, Michael addition, homocysteine-norbornene cross-conjugation. In any embodiment of the method of manufacturing, the pharmaceutical agent may be a radiosensitizer. In any embodiment of the method of manufacturing, the pharmaceutical agent may be selected from the list consisting of doxorubicin, topotecan, and temozolomide.

In any embodiment of the method of manufacturing, the pharmaceutical agent may comprise about 1 to about 1,000 μg aggregate mass per tablet. The pharmaceutical formulation may comprise about 1, 2, 3, 4, 5, 10, 15, 20, 50, 100, 200, 300, 400, 500, 600, 700, 800, 900, 1,000 and up to 3,000 μg aggregate mass per tablet. In an embodiment, the pharmaceutical agent may comprise from about 3 μg up to 25 μg aggregate mass per tablet.

In any embodiment of the method of manufacturing, the tablet may be adapted to fit in or on the microbot. In any embodiment of the method of manufacturing, the tablet may be substantially cylindrical or substantially spheroid. In any embodiment of the method of manufacturing, the tablet may be cylindrical or substantially cylindrical. In any embodiment of the method of manufacturing, the tablet may be spheroid, substantially spheroid, or hemispheroid. In any embodiment of the method of manufacturing, the tablet may have a length/major axis dimension of about 2 mm and a diameter/minor axis dimension of about 1 mm. In any embodiment of the method of manufacturing, the tablet may have a length/major axis dimension of about 1 mm and a diameter/minor axis dimension of about 0.85 mm. The length/major axis dimension may be 50 nm, 55 nm, 60 nm, 70 nm, 80 nm, 90 nm, 100 nm, 150 nm, 200 nm, 300 nm, 400 nm, 500 nm, 750 nm, 1000 nm (1 μm), 1.1 μm, 1.2 μm, 1.3 μm, 1.4 μm, 1.5 μm, 1.6 μm, 1.7 μm, 1.8 μm, 1.9 μm, 2.0 μm, 5.0 μm, 10 μm, 15 μm, 20 μm, 25 μm, 30 μm, 40 μm, 50 μm, 75 μm, 100 μm, 200 μm, 300 μm, 400 μm, 500 μm, 600 μm, 700 μm, 850 μm, 800 μm, 900 μm, 1000 μm (1 mm), 1.1 mm, 1.2 mm, 1.3 mm, 1.4 mm, 1.5 mm, 1.6 mm, 1.7 mm, 1.8 mm, 1.9 mm, 2.0 mm, 2.5 mm, 3.0 mm, 3.5 mm, 4.0 mm, 5.0 mm, 6.0 mm, 7.0 mm, 8.0 mm, 9.0 mm, or 10.0 mm (1 cm). The width/diameter/minor axis dimension may be 50 nm, 55 nm, 60 nm, 70 nm, 80 nm, 90 nm, 100 nm, 150 nm, 200 nm, 300 nm, 400 nm, 500 nm, 750 nm, 1000 nm (1 μm), 1.1 μm, 1.2 μm, 1.3 μm, 1.4 μm, 1.5 μm, 1.6 μm, 1.7 μm, 1.8 μm, 1.9 μm, 2.0 μm, 5.0 μm, 10 μm, 15 μm, 20 μm, 25 μm, 30 μm, 40 μm, 50 μm, 75 μm, 100 μm, 200 μm, 300 μm, 400 μm, 500 μm, 600 μm, 700 μm, 800 μm, 850 μm, 900 μm, 1000 μm (1 mm), 1.1 mm, 1.2 mm, 1.3 mm, 1.4 mm, 1.5 mm, 1.6 mm, 1.7 mm, 1.8 mm, 1.9 mm, 2.0 mm, 2.5 mm, 3.0 mm, 3.5 mm, 4.0 mm, 5.0 mm, 6.0 mm, 7.0 mm, 8.0 mm, 9.0 mm, or 10.0 mm (1 cm).

In any embodiment of the method of manufacturing, the tablet may further comprise at least one pharmaceutically acceptable binder, carrier, and/or excipient. In any embodiment of the method of manufacturing, the at least one pharmaceutically acceptable binder, carrier, and/or excipient may be selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone. In any embodiment of the method of manufacturing, the tablet may further comprise a pharmaceutically acceptable coating material.

In any embodiment of the method of manufacturing, the tablet may conform to USP905 consistent uniformity requirements, as specified in the Stage 6 Harmonization monograph (1 Dec. 2011) (www.usp.org/sites/default/files/usp/document/harmonization/gen-method/q0304_stage_6_monograph_25_feb_2011.pdf) which is incorporated herein by reference in its entirety.

In an aspect, the present disclosure provides a method of manufacturing a tablet to be carried as payload in or on a microbot, comprising the steps of: (1) preparing a premix consisting of about 90 parts doxodubicine, about 450 parts hydrous lacrosse, about 60 parts polyvinyl alcohol, about 35 parts purified water, about 6 parts fumed silica, and about 6 parts magnesium stearate; (2) raising the premix to about 50° C. for a duration of about 16 hours; (3) passing the premix through a 30 mesh standard screen; and (4) pressing the premix into tablets.

In another aspect, the present disclosure provides another method of manufacturing a tablet to be carried as payload in or on a microbot, comprising the steps of: (1) preparing a premix consisting of about 444 parts doxodubicine, about 545 parts hydrous lactose, and about 10 parts magnesium stearate; (2) dry blending the premix; and (3) placing the premix into a tablet press configured to press spheroidal tablets having a length of about 1 mm and average diameter of about 0.85 mm.

In still another aspect, the present disclosure provides another method of manufacturing a tablet to be carried as payload in or on a microbot, comprising the steps of: (1) suspending the at least one biologic- or cell-based therapeutic in a hydrogel matrix, the hydrogel matrix comprising NorHA; and (2) packing the cargo into dimensions such that the tablet may fit on or in the microbot.

1. A pharmaceutical formulation, adapted to be carried as payload in or on a microbot, comprising:
a tablet comprising a therapeutically effective dose of a pharmaceutical agent and having dimensions such that the tablet may fit on or in the microbot.

2. The pharmaceutical formulation of claim 1 wherein the pharmaceutical agent is a small molecule drug, a peptide, a peptoid, cells, or a biologic drug, or any combination thereof.

3. The pharmaceutical formulation of claim 2 wherein the biologic drug or cells are suspended in a hydrogel matrix.

4. The pharmaceutical formulation of claim 3 wherein the hydrogel matrix consists of a cross-conjugated homocysteine-norbornene complex.

5. The pharmaceutical formulation of claim 1 wherein the pharmaceutical agent comprises a radiosensitizer.

6. The pharmaceutical formulation of claim 1 wherein the pharmaceutical agent is selected from the list consisting of doxorubicin, topotecan, and temozolomide.

7. The pharmaceutical formulation of claim 1 wherein the pharmaceutical agent comprises about 1 to about 3,000 μg aggregate mass per tablet.

8. The pharmaceutical formulation of claim 1 wherein the pharmaceutical agent comprises about 3 to about 25 μg aggregate mass per tablet.

9. The pharmaceutical formulation of claim 1 wherein the tablet is formulated by wet granulation, dry granulation, or melt extrusion.

10. The pharmaceutical formulation of claim 1 wherein the tablet is adapted to fit in the microbot.

11. The pharmaceutical formulation of claim 1 wherein the tablet is substantially cylindrical or substantially spheroid.

12. The pharmaceutical formulation of claim 10 wherein the tablet is substantially cylindrical.

13. The pharmaceutical formulation of claim 10 wherein the tablet is substantially spheroid.

14. The pharmaceutical formulation of claim 1 wherein the tablet has a length/major axis dimension of about 2 mm and a diameter/minor axis dimension of about 1 mm.

15. The pharmaceutical formulation of claim 1 wherein the tablet has a length/major axis dimension of about 1 mm and an average diameter/minor axis dimension of about 0.85 mm.

16. The pharmaceutical formulation of claim 1 wherein the tablet further comprises at least one pharmaceutically acceptable binder, carrier, and/or excipient.

17. The pharmaceutical formulation of claim 16 wherein the at least one pharmaceutically acceptable binder, carrier and/or excipient is selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone.

18. The pharmaceutical formulation of claim 1 wherein the pharmaceutical agent comprises at least one biologic drug.

19. The pharmaceutical formulation of claim 18 wherein the at least one biologic drug is suspended in an isotonic lyophilized polyplex.

20. The pharmaceutical formulation of claim 19 wherein the isotonic lyophilized polyplex is selected from the group consisting of: poly-L-lysine, phosphorylcholine-modified polyethyleneimine, and PEGylated polyethyleneimine.

21. The pharmaceutical formulation of claim 1 wherein the tablet further comprises a pharmaceutically acceptable coating.

22. The pharmaceutical formulation of claim 1 wherein the pharmaceutical formulation conforms to USP905 consistent uniformity requirements.

23. A therapeutic delivery system comprising a microbot, having a dimension ranging from 50 nm to 1 cm, and having a motility component, and carrying a therapeutic cargo comprising the pharmaceutical formulation of claims 1-22.

24. A method of manufacturing a tablet adapted to serve as a cargo on a microbot, the method comprising the steps of:
forming a premix, the premix comprising a mixture comprising a therapeutically effective dose of a pharmaceutical agent;
shaping the premix into a tablet having dimensions such that the tablet may fit on or in the microbot.

25. The method of manufacturing a tablet of claim 24 wherein the shaping is achieved by wet granulation, dry granulation, or melt-extrusion.

26. The method of manufacturing a tablet of claim 24 wherein the pharmaceutical agent is a small molecule drug, a peptide, a peptoid, cells, or a biologic drug, or any combination thereof.

27. The method of manufacturing a tablet of claim 26 wherein the biologic drug or cells are suspended in a hydrogel matrix.

28. The method of manufacturing a tablet of claim 27 wherein the hydrogel matrix is based on ‘click’ chemistry.

29. The method of manufacturing a tablet of claim 24 wherein the pharmaceutical agent comprises a radiosensitizer.

30. The method of manufacturing a tablet of claim 24 wherein the pharmaceutical agent is selected from the list consisting of doxorubicin, topotecan, and temozolomide.

31. The method of manufacturing a tablet of claim 24 wherein the pharmaceutical agent comprises about 1 to about 3,000 μg aggregate mass per tablet.

32. The method of manufacturing a tablet of claim 24 wherein the tablet is adapted to fit in or on the microbot.

33. The method of manufacturing a tablet of claim 24 wherein the tablet is substantially cylindrical or substantially spheroid.

34. The method of manufacturing a tablet of claim 32 wherein the tablet is substantially cylindrical.

35. The method of manufacturing a tablet of claim 32 wherein the tablet is substantially spheroid.

36. The method of manufacturing a tablet of claim 24 wherein the tablet has a length/major axis dimension of about 2 mm and a diameter/minor axis dimension of about 1 mm.

37. The method of manufacturing a tablet of claim 24 wherein the tablet has a length/major axis dimension of about 1 mm and an average diameter/minor axis dimension of about 0.85 mm.

38. The method of manufacturing a tablet of claim 24 wherein the tablet further comprises at least one pharmaceutically acceptable binder, carrier, and/or excipient.

39. The method of manufacturing a tablet of claim 38 wherein the at least one pharmaceutically acceptable binder, carrier and/or excipient is selected from the group consisting of: polyvinyl pyrrolidone, dextrin and/or relevant dextrin derivatives, polylactic acid, polyglycolic acid, mixed polymer(s) of lactic and glycolic acid (PLGA), hydrous lactose, polyvinyl alcohol, water, fumed silica, magnesium stearate, hyaluronic acid, agarose, collagen, chitosan, trehalose, sucrose, lactosucrose, dextran, hydroxypropyl betadex, and povidone.

40. The method of manufacturing a tablet of claim 24 wherein the pharmaceutical agent comprises at least one biologic drug.

41. The method of manufacturing a tablet of claim 40 wherein the at least one biologic drug is suspended in an isotonic lyophilized polyplex.

42. The method of manufacturing a tablet of claim 41 wherein the isotonic lyophilized polyplex is selected from the group consisting of: poly-L-lysine, phosphorylcholine-modified polyethyleneimine, and PEGylated polyethyleneimine.

43. The method of manufacturing a tablet of claim 24 further comprises coating the tablet in a pharmaceutically acceptable coating material.

44. The method of manufacturing a tablet of claim 24 wherein the tablet conforms to USP905 consistent uniformity requirements.

45. A method of manufacturing a tablet adapted to be carried as payload in or on a microbot, the method comprising the steps of:
preparing a premix consisting of about 90 parts doxodubicine, about 450 parts hydrous lacrosse, about 60 parts polyvinyl alcohol, about 35 parts purified water, about 6 parts fumed silica, and about 6 parts magnesium stearate;
raising the premix to about 50° C. for about 16 hours;
passing the premix through a 30 mesh standard screen; and
pressing the premix into tablets.

46. A method of manufacturing a tablet adapted to be carried as payload in or on a microbot, the method comprising the steps of:
preparing a premix consisting of about 444 parts doxodubicine, about 545 parts hydrous lactose, and about 10 parts magnesium stearate;
dry blending the premix; and
placing the premix into a tablet press configured to press spheroidal tablets having a length of about 1 mm and average diameter of about 0.85 mm.

47. A method of manufacturing a cargo carrying at least one biologic- or a cell-based therapeutic, adapted to be carried as payload in or on a microbot, the method comprising the steps of:
suspending the at least one biologic- or cell-based therapeutic in a hydrogel matrix, the hydrogel matrix comprising NorHA; and
packing the cargo into dimensions such that the tablet may fit on or in the microbot.
